(19) United States
(12) Patent Application Publication
(10) Pub. No.: US 2007/0123533 A1
(43) Pub. Date: May 31, 2007

(54) New process for preparing an optically pure 2-morpholinol derivative

(75) Inventors: Philippe Adam, Maxeville (FR); Olivier Ludemann-Hombourger, Pompey (FR); Elias Ndzie, Velaine en Haye (FR); David Simon Ross, Tonbridge (GB); Mireille Schaeffer, Plobsheim (FR); Cristina Suteu, Illkirch (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 10/577,417

(22) PCT Filed: Oct. 25, 2004
(86) PCT No.: PCT/EP04/12093

§ 371 Date: Apr. 26, 2006

(30) Foreign Application Priority Data
Oct. 27, 2003 (GB) 0325055.2

(51) Int. Cl. C07D 265/32

(52) U.S. Cl. 514/238.2; 544/170

(57) ABSTRACT

The present invention relates to a process for preparing optically pure (+)-(2S,3S)-2-(3-chlorophenyl)-3,5,5-trimethyl-2-morpholinol and pharmaceutically acceptable salts and solvates from a mixture of (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol. The process utilizes continuous chromatography, including techniques like Multi Column Chromatography (MCC), VARICOL, and Cyclojet.

### BACKGROUND OF THE INVENTION

1. Field of the Invention

The present invention relates to a process for preparing optically pure (+)-(2S,3S)-2-(3-chlorophenyl)-3,5,5-trimethyl-2-morpholinol and pharmaceutically acceptable salts and solvates thereof from a mixture of (+)(2S,3S-2-(3-chlorophenyl)-3,5,5-trimethyl-2-morpholinol and (−)-(2R,3R)-2-(3-chlorophenyl)-3,5,5-trimethyl-2-morpholinol, for example, a racemic mixture.

2. Description of the Prior Art

The separation of enantiomerically pure or optically enriched enantiomers from a mixture of two enantiomers has traditionally been performed by a diastereoisomer salt formation, a crystallization method, or an enzymatic method; such methods are well known in the art. Chiral chromatography is well known as an analytical technique and is also a useful means for separating both enantiomers at the preparative scale. Separation of optically enriched enantiomers using batch chromatography; however, has rarely been advanced to the commercial production of a specific pharmaceutical compound due to a number of technical criteria required. Namely batch chromatography gives rise to a high dilution of the initial feed concentration, which leads to a large quantity of eluent being required and the inefficient use of the chiral stationary phase. Consequently, the concentration of the desired compound in the eluent is, therefore, low and this requires large amounts of energy to isolate the desired material and recovery of the solvent for re-use. Continuous chromatography negates some of these disadvantages, allowing higher efficiency in terms of separated product per amount of stationary phase compared to batch chromatography and generally with much lower solvent usage, therefore requiring less energy for recovery.

For instance, examples of continuous chromatography are liquid chromatography technologies known by the names of Multi-Column Chromatography (MCC), Cyclojet, Simulated Moving Bed (SMB), and VARICOL®. MCC is a general term encompassing SMB and VARICOL®. The concept of SMB was patented in the early 1960's (U.S. Pat. Nos. 2,957,927, 2,985,589, and 3,291,726) and has been used for some time in the petrochemical industry (U.S. Pat. Nos. 3,205,166 and 3,310,486). U.S. Pat. Nos. 5,434,298, 5,434,299 and 5,498,752 also relate to SMB processes. U.S. Pat. No. 5,518,625 relates to the use of an SMB process for separation under low retention capacity conditions. The concept of Cyclojet is also well-known but it has not been demonstrated on the large scale. Lately the VARICOL® system has been used and is described in U.S. Pat. Nos. 6,136,198, 6,375,839 and 6,413,419. VARICOL® is a non-SMB process which is a variation on MCC technology that offers several advantages, such as higher throughput in terms of more feed processed and generally lower solvent consumption; in other words, MCC technology can produce a more consistent product quality for fixed productivity and solvent consumption (see A. Toumi et al., J. Chrom., Vol. 1006, (2003), pages 15-31 and Z. Zhang et al., AlChE Journal, December 2002, Vol. 48, No. 12, 2800-2816). Published application WO 00/25885 relates to VARICOL® technology, and published application U.S. 2002/0014458 A1 relates to optimisation of an SMB process. U.S. Pat. No. 6,107,492, and published applications WO 99/57089, WO 03/006449, WO 03/037840, WO 03/051867, WO 03/072562 and WO 2004/046087 relate to processes for the preparation of specific compounds.

The compound (+)-(2S,3S)-2-(3-chlorophenyl)-3,5,5-trimethyl-2-morpholinol and pharmaceutically acceptable salts and solvates thereof, and pharmaceutical compositions comprising the same are used in treating numerous diseases or disorders such as depression, attention deficit hyperactivity disorder (ADHD), obesity, migraine, pain, sexual dysfunction, Parkinson's disease, Alzheimer's disease, or addiction to cocaine or nicotine-containing (including tobacco) products. Several literature references describe the preparation of either the (+)(2S,3S) or (−)-(2R,3R)-enantiomers from the racemate (+/−)-(2R*, 3R*)-2-(3-chlorophenyl)-3,5,5-trimethyl-2-morpholinol. For instance, reference is made to U.S. Pat. No. 6,342,496 B1, issued to Jerussi et al. on Jan. 29, 2002; U.S. Pat. No. 6,337,328 B1, issued to Fang et al. on Jan. 8, 2002; U.S. Pat. No. 6,391,875 B1, issued to Morgan et al. on May 21, 2002; U.S. Pat. No. 6,274,579 B1, issued to Morgan et al. on Aug. 14, 2001; U.S. Patent Application Publication Nos. 2002/0052340 A1, 2002/0052341 A1, and 2003/0027827 A1; as well as WO 01/62257 A2. However, none of these references utilizes a continuous chromatographic technique for purification of the racemate.

Continuous chromatography techniques, when performed correctly, allow higher efficiency in terms of separated product per amount of stationary phase with substantially lower solvent costs over classical batch chromatography and can become comparable to traditional classical separation in terms of cost. However, the set up of a robust and efficient continuous chromatography system is difficult and has hitherto been unknown for preparing optically pure (+)-(2S,3S)-2-(3-chlorophenyl)-3,5,5-trimethyl-2-morpholinol.

### SUMMARY OF THE INVENTION

The present invention relates to a process for preparing optically pure or enriched (+)-(2S,3S)-2-(3-chlorophenyl)-3,5,5-trimethyl-2-morpholinol from a mixture of (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol which can be a racemic or non-racemic mixture. The separation, by either a batch chromatography system and/or a continuous chromatography system, utilizes a chiral stationary phase (CSP), such as amylose tris-3,5-dimethylphenylcarbamate (CHIRALPAK® AD) or a chemically modified form thereof (CHIRALPAK® T101). This continuous chromatography includes systems such as MCC, VARICOL®, and Cyclojet. VARICOL® is the preferred method in terms of amounts of feed capable of being processed, robust operating parameters and consistent product quality.

Coupling continuous chromatography with crystallization techniques can bring benefits (H. Lorenz et al., Journal of Chromatography A, Vol. 908 (2001), pages 201-214). The performance (i.e. the productivity from a chromatographic system which is generally quoted in kilograms of racemate processed per kilograms CSP per day (kg racemate/kg CSP/day)) can strongly depend on the specification of the optical purity at the outlet of the chromatographic system. This coupling is intended to increase the productivity of the unit by producing a slightly lower optical purity. Crystallization can then be used as a further optical purification process e.g. starting with a mixture of enantiomers enriched in (+)-(2S,3S)-2-(3-chlorophenyl)-3,5,5-trimethyl-2-morpholinol (chiral purity 92% peak area ratio (PAR)) crystals of pure (>99.5%) (+) -(2S,3S)-2-(3-chlorophenyl)-3,5,5-trimethyl-2-morpholinol can be obtained with a recovery yield of 92% of the theoretical recoverable amount of pure enantiomer. The mother liquors obtained from this show the same composition as the eutectic point (85% purity). Crystallization as a chemical purification process is also highlighted. A highly pure (99.5%) thick slurry solution of (+)-(2S,3S)-2-(3-chlorophenyl)-3,5,5-trimethyl-2-morpholinol that is filtered and washed with cold acetonitrile will give pure (+)-(2S,3S)-2-(3-chlorophenyl)-3,5,5-trimethyl-2-morpholinol with most impurities remaining in the mother liquor.

The chiral purity of the desired enantiomer is at least 85% and generally ranges between 98% and 99.9% with a recovery of the required enantiomer of at least 90%, generally about 96%. Racemization of the unwanted (−)-(2R,3R)-2-(3-chlorophenyl)-3,5,5-trimethyl-2-morpholinol can also be coupled with the present purification process and recycled back into the feedstream. This will significantly reduce the required amount of racemate required to produce the desired (+)(2S,3S)-2-(3-chlorophenyl)-3,5,5-trimethyl-2-morpholinol.

### DETAILED DESCRIPTION OF THE INVENTION

The present invention includes a process for preparing optically enriched (+)-(2S,3S)-2-(3-chlorophenyl)-3,5,5-trimethyl-2-morpholinol and pharmaceutically acceptable salts and solvates thereof. Mixtures of (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol which can be optically enriched in accordance with the present invention can be produced by various methods known in the art. The mixtures produced by such processes will usually be racemic mixtures comprising a 50/50 mixture of (+)-(2S,3S-2-(3-chlorophenyl)-3,5,5-trimethyl-2-morpholinol and (−)-(2R,3R)-2-(3-chlorophenyl)-3,5,5-trimethyl-2-morpholinol. However, the present invention can be used to optically enrich others mixtures such as those containing greater than 50% of (+)-(2S,3S)-2-(3-chlorophenyl)-3,5,5-trimethyl-2-morpholinol and substantial amounts of (−)-(2R,3R)-2-(3-chlorophenyl)-3,5,5-trimethyl-2-morpholinol. The feedstock for the process will comprise both (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol identified above and a solvent suitable for the continuous chromatography process. Undesired chemicals (including impurities), for instance impurities present from the synthesis of the original mixture, present in the feedstock may be removed prior to subjecting the feedstock to the continuous chromatography.

After (+)-(2S,3S)-2-(3-chlorophenyl)-3,5,5-trimethyl-2-morpholinol is purified by the continuous chromatography process and possibly subjected to further purification by crystallization of the present invention, it may be converted into pharmaceutically acceptable salts thereof or solvates thereof, particularly those of U.S. Pat. No. 6,342,496 B1, U.S. Pat. No. 6,337,328 B1, U.S. Pat. No. 6,391,875 B1, U.S. Pat. No. 6,274,579 B1, U.S. Patent Application Publication Nos. 2002/0052340 A1, 2002/0052341 A1, and 2003/0027827 A1, as well as WO 01/62257 A2.

The MCC, as described in U.S. Pat. No. 2,985,589 issued to Broughton, using a chiral stationary phase is used to provide (+)-(2S,3S)-2-(3-chlorophenyl)-3,5,5-trimethyl-2-morpholinol in 80-100% enantiomeric excess, preferably in at least 90% enantiomeric excess.

Suitably the MCC is carried out in a four zone cascade apparatus which is one of the most efficient implementations of the MCC process, see in U.S. Pat. No. 2,985,589.

The optimal conditions for an MCC separation are generally identified by analyzing elution profiles obtained from HPLC (high performance liquid chromatography). Important parameters are: loadability of the support, mobile phase strength, selectivity, temperature and feed solubility. The optimization of these parameters aids in identifying conditions for cost-effective separations. The methodology used to identify the conditions for MCC operation is discussed and exemplified in the Journal of Chromatography A, Vol. 702, (1995), pages 97-112.

The preferred MCC procedure may be used as part of a two-stage “enriching-polishing” procedure in which a first pass through MCC is used for enrichment followed by another separation technique to enhance the enrichment. The second stage may be another MCC stage. Alternatively, the second stage may be a different procedure, for example HPLC or crystallization.

The mobile phase may be a single component or a mixture of C₅-C₇ alkanes (especially hexane and heptane), C₁-C₃ alkanols (especially methanol, ethanol, and 2-propanol), methyl tert-butyl ether (MTBE), ethyl acetate, acetone, acetonitrile, most preferably the mobile phase is a combined eluent of acetonitrile and isopropanol. The preferred ratio of acetonitrile/isopropanol is between 93/7% v/v to 99/1% v/v, preferably between 95/5% v/v to 97/3% v/v, most preferably 95/5% v/v. In another embodiment the mobile phase is a combined eluent of acetonitrile and methanol, or acetonitrile and ethanol. Also, pure supercritical fluids (SCF), and SCF with alcohols can be used. In addition to the eluents named above, small amounts of base(s) (such as diethyl amine) or acid(s) (such as HCl) can be added as would be known to those in the art. Typically, the amount is less than 2% w/w based on the total weight of solvent. As used herein, the terms “hexane” and “heptane” refer to straight chain, and branched chain isomers thereof.

Chiral chromatography of the racemate (+/−)-(2R*, 3R*)-2-(3-chlorophenyl)-3,5,5-trimethyl-2-morpholinol provides the (+)(2S,3S) enantiomer in at least 90%, preferably greater than 95%, enantiomeric excess, preferably by MCC chromatography using CHIRALPAK® AD as the chiral stationary phase, and acetonitrile or acetonitrile/isopropanol as the mobile phase.

Using the continuous chromatography technique, the purity of the desired enantiomer ranges between 98% and 99.5%, with a recovery of the required enantiomer of 96%. Other embodiments of the present invention include coupling the continuous chromatography technique with a post separation crystallization of the desired enantiomer to achieve the desired purity and recovery. Other embodiments include the racemization of the unwanted enantiomer and recycling of the new mixture into the feedstock.

Optional Crystallization

The enantiomeric excess (e.e.) in the raffinate and/or extract is usually above 90%, preferably above 95% or even more preferably above 98%. However, since it is possible to improve e.e. by a subsequent crystallization step, an e.e. of as low as 60% in the raffinate and/or extract is sufficient to be able to prepare compounds according to the present invention. It is also possible to improve the e.e. by converting a compound into a base addition salt thereof and crystallize the salt.

In one embodiment, the e.e. in the raffinate and/or the extract is 60% and above, preferably above 70% and even more preferably above 80%. The e.e. thereafter is improved by subsequent crystallization, optionally with a pre-conversion of the compound into a base addition salt.

The purpose behind the post separation crystallization is to allow for a higher throughput with a resultant decrease in purity. This decrease in purity can then be corrected or offset by performing a subsequent crystallization.

Optional Racemization

Depending upon the desired enantiomer, either the extract or raffinate flow is undesired. In the present instance and in the following examples, it is the raffinate that ultimately contains the desired enantiomer and the extract that contains the undesired enantiomer. However, it is both unnecessary and wasteful to simply discard this extract. Rather, the undesired enantiomer may be racemized, either chemically or otherwise. It is therefore possible to recycle the extract into the feed stream by first performing a racemization. This will both recycle the extract and reduce the amount of new racemic feed required.

In the present instance, the chemical structure of the compound of interest has a chiral carbon atom with a hydrogen atom attached to it. This hydrogen atom is relatively labile due to its vicinal environment and racemization could be expected under the influence of a basic or acidic agent.

Several methods of racemization are well known. They usually require assistance of external agents (acidic and basic) or sometimes a simple refluxing of a solution of pure enantiomer in a solvent (which is usually protic). This last option has the advantage of not introducing an external agent which has to be removed before recycling the racemized enantiomer in the feed stream.

Due to regulatory requirements, the original racemate and newly formed racemate generated via racemization should show essentially similar impurity profiles. However, any additional impurity in the newly formed racemate can be eliminated by recrystallizing the newly formed racemate and therefore the original impurity profile of the feed racemate can be matched.

Amongst the possible solvents that can be used as racemizing agents, preferably the solvent has a boiling point of at least 50° C. More preferably, the solvent has a boiling point of 55-110° C. Most preferably, the solvent is at least one selected from the following: alkyl acetate, such as methyl acetate, ethyl acetate (sometimes referred to herein as “EtOAc”), isopropyl acetate, propyl acetate, butyl acetate; dialkyl ketone such as 2,4-dimethyl-3-pentanone, 3-methyl-2-butanone, 2-butanone and 4-methyl-2-pentanone; a nitrile such as acetonitrile and propionitrile; a monoalcohol such as methanol or isopropanol; a polyalcohol such as diethylene glycol; and acidic mixtures such as Water/HCl and Methanol/HCl.

Chiral Stationary Phase (CSP) Adsorbent

The adsorbent in the present invention is preferably a chiral stationary phase. Exemplary chiral stationary phases include cellulose derivatives (e.g., esters or carbamates of cellulose, preferably coated on silica), tartrate phases, Tr-acidic and Tr-basic chiral stationary phases (Pirkle phases), amylose derivatives (e.g., esters or carbamates of amylose, preferably coated on silica), polyacrylamide phases, and the like.

Some commercially available chiral stationary phases include microcrystalline cellulose-triacetate (Tradename MCTA or CTA-1), cellulose tris(phenylcarbamate) (Tradename CHIRACEL OJ), cellulose tris (3,5-dimethylphenylcarbamate) (Tradename CHIRACEL OD), cellulose tribenzoate (Tradename CHIRACEL OB), amylose tris[(S)-methylbenzyl-carbamate] (Tradename CHIRALPAK AS-V), O,O′-bis(4-tert-butyl-benzoyl)-N,N′-diallyl-L-tartardiamide (Tradename KROMASIL CHI-TBB), O,O′-bis(dimethyl-benzoyl)-N,N′-diallyl-L-tartardiamide (Tradename KROMASIL CHI-DMB), and 3,5-dinitrobenzoylphenylclycine (either ionic or covalent bonding) (Tradename DNBPG). Each of the CHIRACEL and CHIRALPAK products are available from Daicel Chemical Industries, Inc. The KROMASIL products were developed by Separation Products at Eka Chemicals. Suitable chiral stationary phases for MCC include those sold by Chiral Technologies under CHIRALPAK® and CHIRALCEL®. CHIRALPAK® AD, an amylose derivative coated onto silica gel, or the chemically modified form thereof (CHIRALPAK® T101) have been found to be particularly suitable. Other available chiral stationary phases (CSPs) are CHIRALCEL® OJ, CHIRALCEL® OD, WHELK-O 1, KROMASIL DNB, KROMASIL TTB, which are sold by Chiral Technologies, Regis Technologies, and Eka Nobel, respectively.

Particularly preferred is a chiral stationary phase that comprises amylose tris (3,5-dimethylphenylcarbamate) coated on a silica-gel substrate in both 10 μm and 20 μm in size (Tradename CHIRALPAK® AD). The 20 μm CHIRALPAK® AD is considered as the material of choice for scaling up enantioselective preparative scale chromatographic separations as it provides sufficient resolution with reduced back pressures to ensure product quality with high productivity.

Pressure and Temperature

The range of pressures in which the separations of products are carried out in liquid and SCF chromatography can range between about 0.1 to 400 MPa, preferably between 0.5 and 30 MPa. The temperature in the columns is generally between −78° C. and 200° C., preferably between about 5-50° C. more preferably between about 15-40° C., most preferably about 25° C.

Selectivity Parameter “α”

The variables which affect the selectivity include the column type, temperature, pressure, feed rate and solvent mixture. In addition, the selectivity can dramatically increase by conditioning the columns prior to separation, i.e., running the mobile phase through the column (with or without analyte) for at least 12 hours, preferably 12-18 hours. Preferably, the variables are selected to give a Selectivity Parameter “α” of greater than 1.1. More preferably, α is greater than 2.0. Most preferably, α is equal to about 2.5, and especially preferably greater than about 2.5.

The selectivity obtained has a strong influence on process productivity. As illustrated in Example 3 below the addition of isopropanol to the acetonitrile mobile phase increased the selectivity over two-fold. The process productivity is almost proportional to

$${( \frac{α - 1}{α} )}^{3} ,$$

but other parameters also have to be considered to select the best chromatographic conditions. The process is also influenced by the retention of the compounds, with tests showing the retention to be maximal at 2-3% isopropanol. However the solubility of the racemate is shown to increase with increasing isopropanol content allowing a more concentrated feed to be injected. Competing effects are in operation here with 5% isopropanol being shown to be optimal and superior to using acetonitrile alone (c.f. 22.5 g/L in pure acetonitrile and 30 g/L in an acetonitrile/isopropanol 95/5 mixture). Also from the standpoint of process operation robustness, since MCC involves continuous operation it is important to avoid any precipitation effects which could halt the system. Using a mobile phase containing isopropanol reduces the likelihood of precipitation occurring and is advantageous over a MCC system operating purely in 100% acetonitrile in which racemate feed and isolated enantiomers are less soluble. As is shown in Example 3 below in using a mixed solvent (acetonitrile/isopropanol) eluent the obtained process throughput has about twice the specific productivity compared to using pure acetonitrile with reduced eluent consumption (c.f. 270L/kg feed of racemate to 313 L/kg feed of racemate) at the same chiral purity but also with increased robustness of process operating parameters.

Certain non-limiting preferred combinations of mobile phase and chiral stationary phase which have acceptable a values are: a) CHIRALPAK® AD 10 μm with acetonitrile; b) CHIRALPAK® AD 20 μm with acetonitrile, 99.9% acetonitrile+0.1% diethyl amine, 95% acetonitrile+5% 2-propanol, or 90% acetonitrile+10% 2-propanol; and c) CHIRALPAK® 50801 20 μm with acetonitrile or 90% n-heptane+10% ethanol. All percentage concentrations are based on v/v %.

### EXAMPLES

The following Examples are provided for a further understanding of the invention; however, the invention is not to be construed as limited thereto.

### Example 1

### MCC Purification of Racemate with CHIRALPAK® AD 20 μm and Pure Acetonitrile Eluent

This example relates to purification using Multi Column Chromatography (MCC). Good target purity and recovery of (+)-(2S,3S)-2-(3-chlorophenyl)-3,5,5-trimethyl-2-morpholinol were obtained using CHIRALPAK AD 20 μm as stationary phase and pure acetonitrile as eluent.

The separation was performed at 25° C. on an MCC (Multi-Column Continuous Chromatography) system fitted with 6 columns in four separation zones (1-2-2-1). The purity specification was 99.0% with a recovery of 96%. A review of the influence of the optical purity of the first eluted enantiomer on productivity revealed that the productivity could be increased by more than 25% when the required purity decreased from 99.6 to 97.8%.

Racemic compound (+/−)-(2R*, 3R*)-2-(3-chlorophenyl)-3,5,5-trimethyl-2-morpholinol was fed at a flowrate of 1.85 mL/min (total concentration of isomers: 20 g/L of acetonitrile) into a MCC system comprised of six columns of 1.0 cm ID by approximately 10 cm length packed with CHIRALPAK AD. Acetonitrile was used as the eluent at a flowrate of 7.25 mL/min. As a result, an extract was obtained at a flowrate of 6.9 mL/min and a raffinate was obtained at a flowrate of 2.2 mL/min. The compounds in the raffinate and extract were recovered as white solids after evaporation of the solvent. The recovery ranged from 96.2-97.3% and the purity ranged from 97.8-99.6%.

The optimized conditions are set in the following Table 1 below after conditioning of the CSP with the feed solution.

TABLE 1
MCC optimized settings

| | |
|---|---|
| Feed Concentration (g/L) | 20 |
| Feed Flow Rate (mL/min) | 1.85 |
| Eluent Flow Rate (mL/min) | 7.25 |
| Extract Flow Rate (mL/min) | 6.9 |
| Raffinate Flow Rate (mL/min) | 2.2 |
| Recycle Flow Rate (mL/min) | 24.7 |
| Switch Period (min) | 0.42 |

Various columns configurations were tested in order to adjust purities and scan for raffinate purities from 98 to 99.5% with 96% recovery. The column configuration is described in the same order as the zones. In other words, if the column configuration is 1-2-2-1, then Zone 1 has 1 column, Zone 2 has 2 columns, Zone 3 has 2 columns and Zone 4 has 1 column. The zones are defined relative to an inlet point and an outlet point.

Zone I: between the eluent and extract points;

Zone II: between the extract and feed points;

Zone III: between the feed and raffinate points; and

Zone IV: between the raffinate and eluent points.

The three more relevant column configurations that were implemented experimentally are shown in Table 2.

TABLE 2
SMB configurations Tested

| | Column | Flowrate (ml/min) | | | | | | Purity (%) | |
|---|---|---|---|---|---|---|---|---|---|
| Trial | config. | Recycle | Feed | Extract | Raffinate | Eluent | Δt (min) | Raf. | Ext. |
| 1 | 2-2-1-1 | 27.7 | 1.6 | 8.4 | 3.7 | 10.5 | 0.45 | 99.6 | 96.2 |
| 2 | 1-2-2-1 | 27.7 | 2.0 | 8.4 | 4.1 | 10.5 | 0.45 | 99.1 | 96.5 |
| 3 | 1-2-2-1 | 27.7 | 2.2 | 8.4 | 4.3 | 10.5 | 0.45 | 97.8 | 97.3 |

The following Table 3 shows a comparison of productivity and eluent consumption.

TABLE 3
Productivity and Eluent Consumption in the tested settings.

| | | | | Eluent |
|---|---|---|---|---|
| | | | Productivity | Consumption |
| Trial | Purity (%) | Recovery (%) | (kg_{feed}/kg_{CSP}*/day) | (L/kg_{feed}) |
| 1 | 99.6 | 96.2 | 1.63 | 378 |
| 2 | 99.1 | 96.5 | 2.04 | 313 |
| 3 | 97.8 | 97.3 | 2.24 | 289 |

*CSP: Chiral Stationary Phase

The purities and recoveries given in Table 3 were those measured after the system had been running for at least 15-20 cycles, so that it had reached steady state.

### Example 2

### Enantiomeric Enrichment Coupling MCC with Crystallization

Considering the separation of the racemate, the experimental results presented in Example 1 showed that the productivity was significantly influenced by the specified purity. For instance, reviewing Table 3 in Example 1, the productivity was reduced by approximately 25% when the specified purity was increased from 97.8% to 99.6%. Accordingly, enantiomeric enrichment via crystallization was achieved on one of the fractions (raffinate) obtained from the MCC of Example 1. The solvent was evaporated from the raffinate to dryness. A white solid was obtained with a purity of about 96.8% (e.e. 93.6%). Enantiomeric enrichment by recrystallization was subsequently performed on this solid using acetonitrile (the same solvent used in the MCC step). Optical purity of the crystals was increased from 96.8% (e.e. 93.6%) to nearly 99.7% (e.e. 99.4%).

Example 2 illustrates that an enriched solution of the target enantiomer can be successfully crystallized to reach a very high final purity. Coupling chromatography and crystallization for the purification of the required enantiomer is therefore a feasible alternative to improve the productivity and reduce the separation cost.

### Example 3

### MCC Purification of Racemate with CHIRALPAK® AD 20 μm and Acetonitrile/2-propanol Eluent Mixture and VARICOL® Optimization

This Example relates to the purification of racemic (+/−)-(2R*, 3R*)-2-(3-chlorophenyl)-3,5,5-trimethyl-2-morpholinol using MCC. The procedure was substantially the same as described in Example 1, except that the eluent of acetonitrile was replaced with an acetonitrile/2-propanol eluent mixture. This resulted in improved selectivity (a=4.53) compared to Example 1 (a=1.92).

The separation was performed on the racemate using CHIRALPAK® AD 20 μm as a stationary phase. The best elution conditions were obtained with acetonitrile/isopropanol 95/5% v/v as an eluent. The separation itself was performed on an MCC (Multi-Column Continuous Chromatography) system fitted with 6 columns (10 mm column diameter, 100 mm length). The purity specification was 99.0% with a recovery of 96% for the (+)(2S,3S)-2-(3-chlorophenyl)-3,5,5-trimethyl-2-morpholinol enantiomer (less retained enantiomer).

The best performance was obtained with a 6-column VARICOL® process. A throughput of 4.6 kg_{feed}/kg_{CSP}/day was achieved.

Various operating conditions were tested in order to adjust raffinate purity at 99.0% with 96% recovery. Table 4 presents the optimized configuration allowing the achievement of a raffinate purity of 99.0% with a recovery of 96% using a feed concentration of 30 g/L.

TABLE 4
Optimization of MCC configuration

| | Column | | Flowrate (mL/min) | | | | | | Purity (%) | |
|---|---|---|---|---|---|---|---|---|---|---|
| Trial No | config. | ΔP (bar) | Recycle | Feed | Extract | Raffinate | Eluent | Δt (min) | Raf. | Ext. |
| 1 | 1-2-2-1 | 20 | 22 | 3 | 10 | 7 | 14 | 0.65 | 99.3 | 96.5 |

The obtained throughput was 4.59 kg_{feed}/kg_{CSP}/day. This result can be compared to the results presented above for the pure acetonitrile eluent of Example 1. A throughput of 2.04 kg_{feed}/kg_{CSP}/day (see Trial 2 of Example 1) was obtained for similar purity and recovery constraints. The applied modification of the eluent composition increased the productivity by 120% with the acetonitrile/isopropanol 95/5% v/v eluent composition compared to the pure acetonitrile eluent composition.

Additional experiments were performed to further optimize the process productivity. A variation of the feed flow rate was performed with a simultaneous adjustment of the other operating flow rates. The objective was to maximize the obtained purity, keeping a yield of 96% for the purified raffinate.

Table 5 illustrates various purity/injected feed flow rates. The selected column configuration was 1-2-2-1.

TABLE 5
Influence of the feed flow rate on the MCC performance

| | Feed | Raff. | | | Eluent |
|---|---|---|---|---|---|
| Trial | flow rate | Purity | Yield | Productivity | consumption |
| No | (mL/min) | (%) | (%) | (kg_{feed}/kg_{CSP}/day) | (L/kg_{feed}) |
| 1 | 3 | 99.3 | 96.5 | 4.59 | 189 |
| 2 | 2.8 | 99.5 | 95.7 | 4.28 | 200 |
| 3 | 3.2 | 91.9 | 96.1 | 4.89 | 179 |
| 4 | 3.5 | 84.8 | 96.2 | 5.35 | 167 |

The obtained results showed that a feed flow rate of 3 mL/min was very close to the maximal injectable amount allowing a raffinate purity of 99%. The raffinate purity dropped rapidly when the feed flow rate was further increased.

Next, the above results were optimized by employing the VARICOL® process. The column configuration was optimized to maximize the process productivity and robustness. Table 6 compares the performance of both MCC and VARICOL® processes:

TABLE 6
Comparison of MCC and VARICOL ® Processes

| | Column | ΔP | Flowrate (mL/min) | | | | | | Purity (%) | |
|---|---|---|---|---|---|---|---|---|---|---|
| Process | config. | (bar) | Recycle | Feed | Extract | Raff. | Eluent | Δt (min) | Raff. | Ext |
| MCC | 1-2-2-1 | 20 | 22 | 2.8 | 10 | 6.8 | 14 | 0.65 | 99.5 | 95.7 |
| MCC | 1-2-2-1 | 20 | 22 | 3 | 10 | 7 | 14 | 0.65 | 99.3 | 96.5 |
| VARICOL ® | 1.5-2.3- | 20 | 22 | 3 | 10.1 | 6.9 | 14 | 0.65 | 99.6 | 95.9 |
| | 1.5-0.7 | | | | | | | | | |

The Flow rates are shown in Table 7 below:

TABLE 7
Flow rates

| | |
|---|---|
| Raffinate | 6.90 |
| Feed | 3.00 |
| Extract | 10.10 |
| Eluent | 14.00 |
| Zone IV | 8.00 |
| Zone III | 14.90 |
| Zone II | 11.90 |
| Zone I | 22.00 |

A review of the results of Table 7 reveals that the VARICOL® feed flow rate was set at 3 mL/min. The obtained purity was equal to 99.6%, whereas the best MCC purity obtained with the same feed flow rate was equal to 99.3% (see Trial 1 in Table 5). The purities obtained with the VARICOL® process configuration were equivalent to the MCC performance obtained with a feed flow rate of 2.8 mL/min (very close purity for both extract and raffinate). Application of the VARICOL® process permitted a better column repartition between the four zones and increased the robustness of the separation compared to the MCC process.

### Example 4

### Enantiomeric Enrichment Coupling MCC with Crystallization Using Mixture of acetonitrile/isopropanol

Using a sample of the racemate and the desired enantiomer ((+)-(2S,3S)-2-(3-chlorophenyl)-3,5,5-trimethyl-2-morpholinol), DSC was performed on a SETARAM DSC 131, with a heating rate of 2K/min. Only the values of the top of the endothermic peaks (end of the melting) were considered for the determination of the phase diagram.

Pure enantiomer (+)-(2S,3S)-2-(3-chlorophenyl)-3,5,5-trimethyl-2-morpholinol had an endothermic peak (onset: 392.5 K, peak: 394.5 K, enthalpy: 26184 J/mol), and the racemate had an endothermic peak (onset: 390.1 K, peak: 392.7 K, enthalpy: 32605 J/mol). The theoretical and experimental values are shown in Table 8 below:

TABLE 8
Theoretical and Experimental Values

| | Temperature | Temperature |
|---|---|---|
| X (mole) | from Eq. I (K) | from Eq. 2 (K) |
| 0.5 | 363.6 | 392.7 |
| 0.55 | 367.5 | 392.6 |
| 0.60 | 371.2 | 391.9 |
| 0.65 | 374.7 | 390.9 |
| 0.70 | 378.0 | 389.3 |
| 0.75 | 381.0 | 387.2 |
| 0.79 | 383.4 | 385.0 |
| 0.80 | 384.0 | 384.2 |
| 0.81 | 384.5 | 383.5 |
| 0.82 | 385.1 | 382.8 |
| 0.85 | 386.8 | 380.1 |
| 0.90 | 389.4 | 374.0 |
| 0.95 | 392.0 | 363.1 |
| 1 | 394.5 | — |

In the above Table 8 all of the values are theoretical except for the value of 394.5 at the bottom of the column for equation 1 and 392.7 at the top of the column equation 2. These two values (shown in bold) were experimentally determined by DSC as mentioned above. A review of these values reveals that the eutectic is located around 0.80 (although it varies between 0.80 and 0.85) where the two liquidus of both the racemate and the enantiomer gather.

Using the information relating to the eutectic, it was now possible to optimize the post separation crystallization step. As discussed above, the chromatographic selectivity can be significantly improved with a 95/5 acetonitrile/IPA eluent composition. The modification of the eluent composition has a significant influence on the crystallization step due to the modification of the product solubility in the new selected solvent. Accordingly, purification by crystallization was carried out on the raffinate obtained at the end of the MCC process of Example 3.

Evaporation of the solvent was performed on about 500 grams of raffinate solution (enantiomeric ratio 97.5/2.5, total concentration of solid 13.33 g/L) and was stopped when traces of solid appeared in the round bottom flask. The obtained suspension (total mass of 57.5 g) was heated up to 70° C. in order to re-dissolve the solid. The obtained solution was subsequently transferred into a thermostated jacket at a temperature of about 15° C. under stirring. Precipitation started after 5 minutes. The suspension was left at this temperature for 2 to 3 hours under stirring.

The theoretical yield of recovery of pure enantiomer is expressed as:
% Recovery=% OP−% Eutectic/100%−% Eutectic
The total amount of solid in the 500 g of the initial solution of raffinate was estimated at 5.16 g based on solubility measurements performed on the raffinate (solubility 10.32 g/kg). The optical purity was estimated at 97.5%, and the highest eutectic composition at x=0.85. Therefore, the theoretical yield of the recovery was 83.3%. That means that the theoretical recoverable amount of pure enantiomer from this solution was 4.30 g.

The white solid was filtered off and dried at 40° C. under vacuum (3.81 g of pure enantiomer O.P.>99.5%). The overall yield was about 74% without any practical optimization; this value was compared with the overall theoretical yield of recovery (83.3%).

A second crop was obtained by further cooling of the mother liquors down to 5° C. for 2 hours under stirring. A white solid was recovered by filtration and dried at 40° C. under vacuum (Yield 0.28 g of enantiomer optical purity 94%). The optical purity of the remaining mother liquor was 89.6%.

These results confirm the fact that when a mixture of enantiomers showing an enantiomeric ratio greater than that of the eutectic composition is recrystallized, there is an enrichment of the solid crystallizing phase while the optical purity of the solution (mother liquors) is decreasing towards to the eutectic composition.

Another experiment was carried out on a raffinate having only 82.2% optical purity. Evaporation of solvent was carried out on about 200 g of raffinate at 82.2% optical purity. About 46.6 g of a concentrated solution was recovered and transferred at around 10° C. A white suspension is obtained on stirring during the cooling down to 5° C. after 5 minutes. The suspension was then left under stirring for about 2 hours at this temperature. A white solid was filtered off (0.64 g), dried at 40° C. under vacuum and analyzed by means of chiral HPLC: the optical purity was equal to 70.6%. The optical purity of the mother liquors was about 91.6%, which is higher than that of the solid obtained by crystallization.

This result confirms the fact that when a mixture of enantiomers showing an enantiomeric ratio lower than that of the eutectic composition is recrystallized, there is an enrichment of the mother liquors while the optical purity of the solid phase is decreased towards the racemate.

### Example 5

### Optimization, and Up Scaling of Purification of Racemate with Crystallization Step

This example relates to the enantioseparation of the racemate by Multi-Column Continuous (MCC) chromatography. The separation was performed using CHIRALPAK® AD 20 μm as the stationary phase eluted with an eluent mixture of acetonitrile/isopropanol 95/5 (v/v). The separation was performed on a Lab-MCC system fitted with 6 columns (25 mm internal diameter, 97 mm length). The optical purity specification was 99.5% with a recovery of 96% for the (+)-(2S,3S)-2-(3-chlorophenyl)-3,5,5-trimethyl-2-morpholinol enantiomer (less retained enantiomer).

The operating conditions were optimized and led to a maximal process productivity of 5 kg_{feed}/kg_{CSP}/day with a VARICOL® process. Coupling MCC with crystallization was also performed. Crystallization was used as a further optical purification process starting with a mixture of enantiomers enriched in the target enantiomer (92.7%). Crystals of pure (+)-(2S, 3S)-2-(3-chlorophenyl)-3,5,5-trimethyl-2-morpholinol were obtained with a recovery yield of 92% of the theoretical recoverable amount of pure enantiomer. The mother liquors obtained showed the same composition as the eutectic point.

Crystallization as a chemical purification process was performed by evaporating a highly pure solution (>99.5%) of (+)-(2S,3S)-2-(3-chlorophenyl)-3,5,5-trimethyl-2-morpholinol until a thick slurry was obtained. The solid obtained by filtration was washed with cold acetonitrile. Pure (+)-(2S,3S)-2-(3-chlorophenyl)-3,5,5-trimethyl-2-morpholinol was obtained and most of the impurities were recovered in the mother liquors.

Racemization of the (+)-(2S,3S)-2-(3-chlorophenyl)-3,5,5-trimethyl-2-morpholinol was also performed using methanol under reflux as a solvent.

The process was designed for the purification of 100 metric tons of racemate per year, taking into account the robustness factor necessary to guarantee both optical purity and productivity of the production process. Considering an optical purity of 99.5%, the separation can be achieved on a 6-column MCC unit with an internal diameter of 600 mm. The chromatographic process can be coupled with a washing step of the purified crystals of the target enantiomer. The unwanted enantiomer can easily be racemized and recycled in the feed stream after a recrystallization step (removal of impurities).

The separation was performed using a 6-column configuration. The columns (2.5 cm id., 9.7 cm length) were packed with CHIRALPAK® AD 20 μm. A first CSP conditioning step was tried before running the test. The system was first run in automatic mode injecting about 30 g of feed, but the retention times in the columns were still lower than expected. A second CSP conditioning was performed by pumping in a recirculation loop a 12 g/L feed solution at 30 mL/min during 60 hours.

The VARICOL® operating conditions were set as follows in Table 9:

TABLE 9
VARICOL ® Operating Conditions
VARICOL ® Initial operating conditions
6 column configuration (1.5/2.3/1.5/0.7)
Dcol = 2.5 cm Lcol = 9.7 cm

| | |
|---|---|
| Feed Concentration (g/L) | 30 |
| Feed Flow Rate (mL/min) | 16.64 |
| Eluent Flow Rate (mL/min) | 77.7 |
| Extract Flow Rate (mL/min) | 56.0 |
| Raffinate Flow Rate (mL/min) | 38.28 |
| Recycle Flow Rate (mL/min) | 122 |
| Switch Period (min) | 0.65 |

The VARICOL® running conditions were then optimized as illustrated in Table 10. Table 10 depicts the optimum yet robust VARICOL® process leading to 99.5% raffinate optical purity with a recovery of 96%:

TABLE 10
Optimization of VARICOL ® Conditions

| | | Flowrate (mL/min) | | | | | |
|---|---|---|---|---|---|---|---|
| Column | | | | Ex- | | | Δt |
| configuration | ΔP (bar) | Recycle | Feed | tract | Raff. | Eluent | (min) |
| 1.5-2.3-1.5-0.7 | 20-25 | 169 | 20 | 77 | 45 | 102 | 0.50 |

The feed flow rate was increased from 20 mL/min as shown above to 23 mL/min (+15%). This resulted in a slight decrease of the recovery (although still>96%), but without any incidence on the raffinate optical purity. When the feed flow rate was set at 25 mL/min, the obtained purity and/or the recovery tended to decrease rapidly. Purity and recovery specifications could not be reached simultaneously, even after adjustment of the internal flow rates. This behavior was confirmed when the feed flow rate was increased to 27 and 30 mL/min. Therefore, the maximum feed flow rate was set at about 23 mL/min.

To increase the overall throughput, a final recrystallization step was performed after the VARICOL® process. The experiment was performed starting with a relatively low raffinate purity (92%). Considering the position of the eutectic (85%), the theoretical recoverable amount of pure enantiomer represents only around 50% of the target enantiomer contained in the initial solution.

1214.4 g of raffinate solution enriched at 92% optical purity with a total concentration of solid of about 5.11% was cooled down from 25° C. (where a clear slightly yellow solution was obtained) to 10° C. at a cooling rate of about 1° C./min. The solution was allowed to stand at 10° C. for 1 hour under stirring to yield the first crystals (nucleation). A further cooling step was performed from 10° C. to 0° C. at 1° C./min cooling rate and the suspension was maintained at this temperature for almost two hours under stirring. The suspension was filtered to yield 25.04 g of dried crystals without washing. The crystals and the mother liquors were analyzed by means of chiral HPLC.

The total amount of solid within the solution was estimated at the beginning at about 62.1 g. Optical purity of this solution was 92%. This meant that the total mass of the enantiomer in excess (S,S-enantiomer) was about 53 g. Considering the position of the eutectic at around 85% enantiomeric ratio, the theoretical total amount of recoverable pure enantiomer was therefore 27.2 g, which was consistent with the recovered amount of dried crystals (25.0 g).

The obtained crystals (no washing step of the crystals) had a purity of 99.6%, whereas analysis of the mother liquor showed that the obtained purity was very close to the estimated eutectic composition.

A chemical purification process following the MCC was also performed. This option was applicable when the purification by MCC yielded almost a pure enantiomer matching the optical purity specifications (e.g. O.P.>99.5%). The resulting raffinate was evaporated to dryness. The solid obtained was washed with cold acetonitrile to remove impurities.

The experimental procedure was that a mixture of 30.4 g of the desired enantiomer obtained by drying a solution of pure raffinate and 50 ml of acetonitrile (purex grade) was slightly warmed (elimination of aggregates) to obtain a thick slurry which was placed at a temperature of around 4° C. for 2 to 3 hours.

A volume of 200 ml of acetonitrile kept at a temperature of about −20° C. for 4 hours was used to wash the solid just after filtration. The thick slurry (slightly yellow) was filtered and the cold solvent was poured onto the crystals and filtered rapidly. After drying at 40° C. under vacuum, 28.5 g of crystals were recovered. The mother liquor after filtration was yellow, whereas the obtained crystals were white, showing that this washing step removed impurities from the crystals. Evaporation of the solvent from the yellow mother liquors produced yellowish solid.

### Example 6

### Optimization of Racemization Step

Pure (+)-(2S,3S)-2-(3-chlorophenyl)-3,5,5-trimethyl-2-morpholinol was tested with various solvents in order to optimize the racemization. Specifically, about 2 g of pure enantiomer ((+)(2S,3S)-2-(3-chlorophenyl)-3,5,5-trimethyl-2-morpholinol) were dissolved into 100 ml (total concentration of about 20 g/L) of solvent (see various solvents below) and heated up to about 60-65° C. under stirring (under reflux). The solution was sampled periodically in order to follow the kinetics of racemization.

Table 11 illustrates the Optical Purity of the racemization of the enantiomer in various solvents or solvent mixtures:

TABLE 11
OP in various solvents or mixtures of solvent during racemization of
(+)-(2S,3S)-2-(3-chlorophenyl)-3,5,5-trimethyl-2-morpholinol

| Solvent or | | Final | |
|---|---|---|---|
| mixture of solvents | Starting OP (%) | OP (%) | Duration (hours) |
| Acetonitrile/IPA (95/5) | >99.8 | 92.8 | 20 |
| Isopropanol | >99.8 | 84.8 | 20 |
| Methanol | >99.8 | 95.3 | 2 |
| Water/HCl | >99.8 | >99.8 | 2 |
| Methanol/HCl | >99.8 | 97.4 | 2 |

A review of Table 11 reveals that racemization occurs in the preferred eluent selected for the purification (acetonitrile/IPA 95/5), but the kinetics is very low (OP still high after 20 hours at 65° C.). This suggests that the optical purity of the purified enantiomer will not be significantly reduced during the final concentration and drying step. Racemization is faster in pure isopropanol, but conversion is still low after 20 hours (30%). Racemization appears to be much more favorable in methanol where 10% conversion is reached after 2 hours. Acidic conditions in methanol do not produce an improvement compared to the result obtained with pure methanol. No racemization is observed with acidic aqueous solvent. The result of this screening shows that, among the racemization agents tested, refluxing (+)(2S,3S)-2-(3-chlorophenyl)-3,5,5-trimethyl-2-morpholinol in pure methanol is the best option for promoting its racemization. Thus, the same results are applicable to the racemization of (−)-(2R,3R)-2-(3-chlorophenyl)-3,5,5-trimethyl-2-morpholinol.

As discussed above, the impurity in the newly formed racemate preferably matches that of the original racemate feedstock. Recrystallizing the newly formed racemate can eliminate this impurity and therefore the original impurity profile can be matched. The recrystallization method is based on the following steps:

The obtained newly formed racemic solution is filtrated and evaporated to dryness. The obtained solid is then dissolved in acetonitrile (20 mL for 2 g of solid) at 60° C. This will produce a yellowish solution which is cooled to 4° C. and stored for 72 hours. This is then followed by filtration.

### Example 7

### MCC Purification of Racemate with CHIRALPAK® T101 20 μm and acetonitrile/2-propanol Eluent Mixture

This Example relates to the purification of racemic (+/−)-(2R*, 3R*)-2-(3-chlorophenyl)-3,5,5-trimethyl-2-morpholinol using MCC. The procedure was substantially the same as described in Example 3, except that the CSP was changed to CHIRALPAK T101. The separation itself was performed on an MCC (Multi-Column Continuous Chromatography) system fitted with 8 columns (10 mm column diameter, 100 mm length).

Various operating conditions were tested. Table 12 presents the optimized configuration allowing the preparation of the (+)-(2S,3S)-2-(3-chlorophenyl)-3,5,5-trimethyl-2-morpholinol enantiomer (less retained enantiomer) with a chemical purity of 99.1% and an enantiomeric excess of 99.7%.

TABLE 12

| | |
|---|---|
| Column | Flowrate (mL/min) |

| config. | Feed | Extract | Raffinate | Eluent | Δt (min) |
|---|---|---|---|---|---|
| 2-2-2-2 | 4.15 | 12.96 | 6.39 | 22.60 | 0.75 |

By using these conditions and extrapolating to a Licosep 8-50 equipment, a productivity of around 2.8 kg feed/kg CSP/day could be reached. The SMB parameters would be as follows:

| Final conditions | 8 columns ID 4.8 cm L10 cm | |
|---|---|---|
| Parameters at 35 bar | Feed concentration 25 g/l | |
| Feed | 71.30 | ml/min |
| Extract | 222.65 | ml/min |
| Raffinate | 109.78 | ml/min |
| Eluent | 261.13 | ml/min |
| ΔT | 45 | s |
| Zone I | 388.26 | ml/min |
| Productivity | 2.81 | kg feed/kg CSP/day |

### Example 8

### MCC Purification of Racemate with CHIRALPAK® T101 20 μm and acetonitrile/2-propanol Eluent Mixture

A total of 2.35 kg of racemate was separated using the Licosep Lab 50 equipment, in VARICOL® mode, on the CHIRALPAK® T101 stationary phase and using 5/95 v/v isopropanol/acetonitrile as the mobile phase. 1.06 kg of the (+)-(2S,3S)-2-(3-chlorophenyl)-3,5,5-trimethyl-2-morpholinol enantiomer (less retained enantiomer), with an optical purity of 97.0% was obtained. The recovery was 89.8% for the raffinate (desired product) with the remainder being eluted in the extract stream. The productivity was 4.16 kg feed/kg CSP/day (24 hr). The solvent consumption was 171 l/kg. This process was not further optimized. The productivity obtained was limited by the maximum flowrate of the feed pump (50 ml/min). The optical purity can be further enhanced by crystallization.

Initial Screening Experiments:

Tables 13A and 13B below summarise the results of single-column screening experiments using different CSP/mobile phase combinations.

The following briefly describes the screening process, with particular reference to the CHIRALPAK and CHIRALCEL CSP's tested. A similar process was used for the other commercially available CSP's tested.

An Agilent 1100 HPLC system is an example of the equipment that may be used for this process, which includes a quaternary G1311A pump for solvent delivery, and a G1313A autosampler for injection. Detection of the column eluent was carried out with an UV DAD detector G1315B. Racemate was chromatographed at a flow rate of 1 ml/min for all the mobile phases described in Tables 13A and 13B at a temperature of 20° C. Separation of the enantiomers was measured by UV at 220 nm.

The terms retention time, capacity factor and selectivity (a) used in Tables 13A and 13B, and how to calculate them, will be well understood by the person skilled in the art (see, for example published patent application WO 2004/046087 at page 7, lines 1 to 4).

Abbreviations used in Tables 13A and 13B are as follows:

| | | |
|---|---|---|
| n-heptane = n-hept | Methyl Acetate = MeOAc | Diethylamine = |
| | | DEA |
| n-hexane = n-hex | Glacial Acetic Acid = HAc | Isopropylalcohol = |
| | | IPA |
| Methanol = MeOH | Tetrahydrofuran = THF | Ethanol = EtOH |
| Ethyl Acetate = | Dichloromethane = MeCl2 | tert-ButylMethyl |
| EtOAc | | Ether = MTBE |

In addition, single-column screening evaluations were also performed on the RU1 and RU2 chiral stationary phases available from Shiseido Fine Chemicals (Japan). The RU2 column packed with 20 μm particles (250 mm×4 mm) using methanol, ethanol, acetonitrile, ethyl acetate and methyl acetate as mobile phases at an elevated column temperature of 50° C. or 60° C. resulted in no separation, with the racemate peaks appearing to be irreversibly bound to the column. The RU1 column gave similar results.

TABLE 13A

| | Particle | | First | Second | |
|---|---|---|---|---|---|
| | size | | peak | peak | Selectivity |
| Column | (μm) | Mobile Phase | Rt (min) | Rt (min) | (α) |
| Chiralpak AD | 20 | 95:5 acetonitrile/EtOH | 6.29 | 6.91 | — |
| Chiralpak AD | 20 | 95:5 acetonitrile/MeOH | 6.28 | 6.96 | — |
| Chiralpak AD | 20 | 90:10 acetonitrile/IPA | 6.00 | 7.65 | 1.55 |
| Chiralpak AD | 20 | acetonitrile | 3.87 | 4.67 | 1.92 |
| Chiralpak AD | 20 | acetonitrile + 0.1% DEA | 3.84 | 4.69 | 2.02 |
| Chiralpak AD | 20 | 95:5 acetonitrile/IPA | 3.63 | 5.84 | 4.53 |
| Chiralpak AD | 20 | 90:10 n-hept/IPA | No separation | | |
| Chiralpak 50801 | 20 | 90:10 n-hept/IPA | No separation | | |
| Chiralpak ASV | 20 | 90:10 n-hept/IPA | No separation | | |
| Chiralcel OD | 20 | 90:10 n-hept/IPA | 4.53 | 4.97 | — |
| Chiralcel OJ | 20 | 90:10 n-hept/IPA | No separation | | |
| Chiralcel OK | 20 | 90:10 n-hept/IPA | 6.53 | 7.12 | — |
| Chiralcel OF | 20 | 90:10 n-hept/IPA | No separation | | |
| Chiralpak AD | 20 | 90:10 n-hept/EtOH | 5.24 | 5.95 | — |
| Chiralpak 50801 | 20 | 90:10 n-hept/EtOH | 5.83 | 6.65 | 1.29 |
| Chiralpak ASV | 20 | 90:10 n-hept/EtOH | No separation | | |
| Chiralcel OD | 20 | 90:10 n-hept/EtOH | No separation | | |
| Chiralcel OJ | 20 | 90:10 n-hept/EtOH | No separation | | |
| Chiralcel OK | 20 | 90:10 n-hept/EtOH | 5.37 | 5.88 | — |
| Chiralcel OG | 20 | 90:10 n-hept/EtOH | 4.42 | 4.74 | — |
| Chiralpak AD | 20 | 50:50 EtOH/MeOH | No separation | | |
| Chiralpak 50801 | 20 | 50:50 EtOH/MeOH | No separation | | |
| Chiralpak ASV | 20 | 50:50 EtOH/MeOH | No separation | | |
| Chiralcel OD | 20 | 50:50 EtOH/MeOH | No separation | | |
| Chiralcel OJ | 20 | 50:50 EtOH/MeOH | No separation | | |
| Chiralcel OK | 20 | 50:50 EtOH/MeOH | No separation | | |
| Chiralpak IA | 5 | 100% acetonitrile | 5.2 | 5.7 | 1.20 |
| Chiralpak IA | 5 | 95:5 acetonitrile/IPA | 4.3 | 4.9 | 1.56 |
| Chiralpak IA | 5 | MeOH | No separation | | |
| Chiralpak IA | 5 | EtOH | No separation | | |
| Chiralpak IA | 5 | 90:10 n-hex/EtOH | 5.87 | 6.6 | 1.26 |

TABLE 13B

| | Particle size | | Capacity factor | Selectivity |
|---|---|---|---|---|
| Column | (μm) | Mobile Phase | (Peak 1) | (α) |
| Kromasil CHI-TBB | 10 | 95:5 n-hept/IPA | 0.98 | 1.05 |
| Kromasil CHI-TBB | 10 | 95:5 n-hept/THF | 1.94 | 1.05 |
| Kromasil CHI-TBB | 10 | 60:40:0.2 n-hept/MTBE/HAc | No separation | |
| Kromasil CHI-TBB | 10 | 90:10:0.15:0.075 n-hept/THF/HAc/DEA | No separation | |
| Kromasil CHI-TBB | 10 | 95:5:0.1 n-hept/THF/TEA | No separation | |
| Kromasil CHI-TBB | 10 | 95:5:0.1 n-hept/THF/DEA | No separation | |
| Kromasil CHI-TBB | 10 | 90:10:0.1 n-hept/DCM/DEA | No separation | |
| Kromasil CHI-TBB | 10 | 94:6:0.1 n-hept/MTBE/DEA | No separation | |
| Kromasil CHI-TBB | 10 | 95:5:0.1 n-hept/EtOAc/DEA | No separation | |
| Kromasil CHI-DMB | 10 | 95:5 n-hept/IPA | 1.02 | 1.10 |
| Kromasil CHI-DMB | 10 | 95:5 n-hept/THF | 1.92 | 1.13 |
| Kromasil CHI-DMB | 10 | 60:40:0.2 n-hept/MTBE/HAc | No separation | |
| Kromasil CHI-DMB | 10 | 90:10:0.15:0.075 n-hept/THF/HAc/DEA | 1.73 | 1.11 |
| Kromasil CHI-DMB | 10 | 95:5:0.1 n-hept/THF/TEA | 1.91 | 1.93 |
| Kromasil CHI-DMB | 10 | 95:5:0.1 n-hept/THF/DEA | 1.66 | 1.13 |
| Kromasil CHI-DMB | 10 | 90:10:0.1 n-hept/DCM/DEA | 2 | 1.09 |
| Kromasil CHI-DMB | 10 | 94:6:0.1 n-hept/MTBE/DEA | 3.24 | 1.13 |

All cited patents, publications, co-pending applications, and provisional applications referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for preparing optically enriched (+)-(2S,3S)-2-(3-chlorophenyl)-3,5,5-trimethyl-2-morpholinol comprising:
subjecting a mixture of (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol to continuous chromatography to resolve (+)-(2S,3S)-2-(3-chlorophenyl)-3,5,5-trimethyl-2-morpholinol from the mixture.

2. The process according to claim 1 wherein said mixture is a racemic mixture.

3. The process according to claim 1, wherein the mixture is passed through an MCC system.

4. The process according to claim 3, wherein the mixture is passed through a VARICOL system.

5. The process according to claim 1, wherein said continuous chromatography comprises contacting an eluent comprising at least one solvent, with a chiral stationary phase, wherein the solvent is selected from the group consisting of C₅-C₇ alkane, C₁-C₃ alkanol, methyl tert-butyl ether, ethyl acetate, acetone and acetonitrile.

6. The process according to claim 5, wherein said eluent is acetonitrile.

7. The process according to claim 5, wherein said eluent is a mixture of acetonitrile and 2-propanol.

8. The process according to claim 7, wherein said acetonitrile to 2-propanol ratio is between 93/7% v/v to 99/1% v/v.

9. The process according to claim 8, wherein said acetonitrile to 2-propanol ratio is between 95/5% v/v to 97/3% v/v.

10. The process according to claim 5, wherein said chiral stationary phase comprises amylose tris-(3,5-dimethylphenylcarbamate).

11. The process according to claim 1, which further comprises crystallizing the (+)-(2S,3S)-2-(3-chlorophenyl)-3,5,5-trimethyl-2-morpholinol obtained from the mixture.

12. The process according to claim 1, wherein said (+)-(2S,3S)-2-(3-chlorophenyl)-3,5,5-trimethyl-2-morpholinol is obtained in a raffinate stream and (−)-(2R,3R)-2-(3-chlorophenyl)-3,5,5-trimethyl-2-morpholinol is obtained in an extract stream.

13. The process according to claim 1 which further comprises racemizing the (−)-(2R,3R)-2-(3-chlorophenyl)-3,5,5-trimethyl-2-morpholinol to form a racemic mixture of (+)-(2S,3S)-2-(3-chlorophenyl)-3,5,5-trimethyl-2-morpholinol and (−)-(2R,3R)-2-(3-chlorophenyl)-3,5,5-trimethyl-2-morpholinol and subjecting the thus formed racemate to continuous chromatography.

14. The process according to claim 13 wherein the racemate is recycled into a feed stream.

15. The process according to claim 13 wherein the racemization is effected in methanol.

16. The process according to claim 1 wherein the (+)-(2S,3S)-2-(3-chlorophenyl)-3,5,5-trimethyl-2-morpholinol is recovered in an amount of at least 90%.
